(19) United States
(12) Patent Application Publication
(10) Pub. No.: US 2015/0045481 A1
(43) Pub. Date: Feb. 12, 2015

(54) Asymmetric Diamine Compounds Containing Two Functional Groups and Polymers Therefrom

(71) Applicant: KOREA ADVANCED INSTITUTE OF SCIENCE AND TECHNOLOGY, Daejeon (KR)

(72) Inventors: Sang Youl Kim, Daejeon (KR); Sun Dal Kim, Daejeon (KR); Im Sik Chung, Daejeon (KR)

(21) Appl. No.: 14/379,302

(22) PCT Filed: Dec. 5, 2012
(86) PCT No.: PCT/KR2012/010447

§ 371 Date: Aug. 18, 2014

(30) Foreign Application Priority Data
Mar. 9, 2012 (KR) 10-2012-0024323

(51) Int. Cl. C07C 217/90; C08G 73/10; C08L 79/08; C07C 213/02

(52) U.S. Cl. CPC C07C 217/90; C07C 213/02; C08G 73/1071; C08L 79/08
USPC 524/104; 564/430; 564/417; 528/182; 524/233; 524/173; 524/113; 524/375; 524/352

(57) ABSTRACT

The present invention relates to a novel diamine compound, wherein two substituents R and R′ are introduced asymmetrically, and a polymer thereof. The polymer may have excellent solubility in the organic solvent and allows for easy processibility after imidization, thus giving proper film maintaining superior properties, such as thermal, mechanical, and optical properties for applications in electrical, electronic, or optical materials.

### CROSS REFERENCE TO RELATED APPLICATIONS AND CLAIM OF PRIORITY

This patent application is a National Phase application under 35 U.S.C. §371 of International Application No. PCT/KR2012/010447, filed Dec. 5, 2012, which claims priority to and the benefit of Korean Patent Application No. 10-2012-0024323, filed Mar. 9, 2012, entire contents of which are incorporated herein by reference.

### BACKGROUND

1. Field of the Invention

The present invention relates to a novel diamine compounds having two substituents, R and R′, introduced asymmetrically to only one side of said diamine compound, processes for producing the diamine compounds, and polymers thereof.

2. Description of the Related Art

Diamine compounds of the present invention can be used as monomers in the synthesis of polyimides, and the polyimide polymers have high solubility in organic solvents at high concentrations, facilitating easy processing. When a polyimide is made as a film, excellent physical properties are maintained, such as thermal, mechanical, and optical properties, therefore, polyimides can be used in applications such as electrical, electronic, or optical materials.

Diamine compound is useful as a monomer in the synthesis of polymer with a backbone having the imide and/or amide groups. However, whereas polyimide or polyamide derived from aromatic diamine has excellent heat resistance and mechanical strength, the polymer may have low solubility, and therefore, low processibility, making polymeric films or fibers therefrom difficult.

For example, U.S. Pat. No. 5,071,997 teaches the aromatic diamine containing asymmetrically fluorinated alkyl group represented by reference formula 1 wherein:

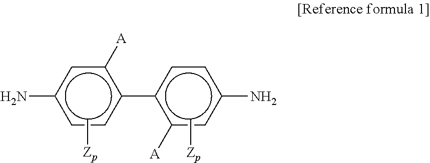

A represents an alkyl group, an aryl group or a substituted aryl group which is fluorinated; Zp represents a functional group substituted into aromatic group. Polyimide manufactured from aforementioned can be used as coating material for micro-electronic devices, gas separation membranes, fiber with high contraction strength and tensile strength.

Additionally, U.S. Pat. No. 5,286,841 teaches asymmetric aromatic diamine compounds, represented by reference formula 2, wherein Rf represents a C1 to C18 linear or branched perfluorinated alkyl group, and soluble aromatic polyimide resulting from condensation of aromatic tetracarboxylic dianhydride and the diamine compounds, and film, fiber or molded parts using the aromatic polyimide.

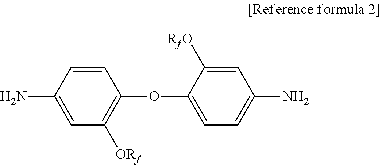

Additionally, KR 10-0562151 B1 and KR 10-0600449 B1 teaches asymmetric diamine compounds, represented as reference formula 3 and reference formula 4 respectively, used in manufacturing highly soluble aromatic polyimide and polyimide thereof.

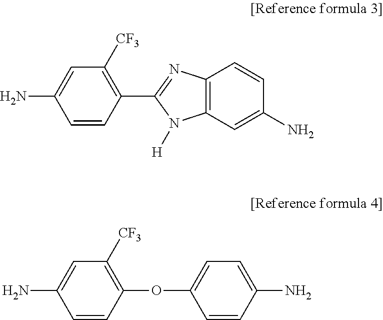 [SMILES: Nc1ccc(-c2nc3ccc(N)cc3[nH]2)c(C(F)(F)F)c1 ; Nc1ccc(Oc2ccc(N)cc2C(F)(F)F)cc1]

In general, polyimide obtained from the condensation of diamine and tetra-carboxylic acid anhydride monomer has excellent heat resistance, mechanical strength, and size stability as well as flame retardancy and electrical insulation. Polyimide has been used in a variety of applications, for example, in electrical and electronic equipment, aerospace equipment, transportation equipment, and the like. Also, several studies are in progress that depend on several application areas.

Among said polyimides, aromatic polyimide has excellent mechanical properties, such as thermal stability, mechanical properties, electrical insulation, chemical resistance, and therefore, said aromatic polyimide are used as representative high performance polymer in the application of interlayer dielectric material in the electronics industry, polymer composite scaffolds, membranes for the separation of gas mixture, polymer electrolyte membrane fuel cell electrolyte, high temperature adhesives, coatings, alignment layer of a liquid crystal display device, and a transparent and flexible electrical, electronic material.

Typical aromatic polyimide is not soluble in an organic solvent after imidization is complete, and rather decomposes before melting has occurred, making the polymer impossible to process. Therefore, the final product is prepared in two steps wherein the first step involves processing the intermediate compound, such as polyamic acid, and the second step involves heat treatment to finish imidization. However, this said preparation method, involving processing polyamic acid as a processible intermediate, followed by curing via heat and chemical treatment to produce final products, creates unstable polyamic acid, the formation of by-products, such as water, in the middle of the curing process which causes deformations and defects in molding articles.

Thus, many studies have been carried out to develop solubilized polyimide with easy processibility as a film or fiber at the final stage after imidization is complete, thereby minimizing deterioration of the physical properties of aromatic polyimide.

A method of manufacturing soluble polyimide is related to reducing interaction between polymer chains, more specifically, introducing a flexible functional group or a bulky functional group onto the polymer backbone, twisting the planar formation of the polymer chains, for example, via using alicyclic monomer, reducing the regularity of molecular structure by introducing asymmetric structure, copolymerizing with compounds having high solubility in organic solvents, etc.

In particular, even though there are not many examples of fully aromatic polyimide, U.S. Pat. No. 7,550,194 B2 teaches fully aromatic polyimide with high solubility and transparency prepared from diamine (2,2′-bis(trifluoromethyl)benzidine), wherein said diamine has two trifluoromethyl groups on biphenyl, represented as reference formula 5.

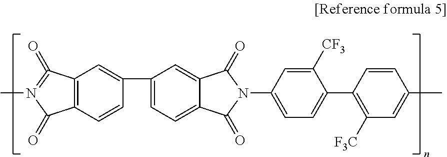

Introduction of perfluoroalkyl group such as bulky trifluoromethyl groups, via carbon-fluorine bond stronger than carbon-hydrogen bond, does not lower heat resistance, while inhibiting several interactions which conventional polyimide has, therefore increasing the solubility of polymer as well as resulting in significantly low water adsorption, dielectric constant, and refractive index, etc, resulting from low polarizability of fluorine atom.

### SUMMARY

In one embodiment, the polyimide is synthesized using a novel diamine which has two functional groups, R and R′, asymmetrically attached to one cyclic group of diamine compounds, thereby allowing the use of the diamine as a monomer in the preparation of polyimide.

More specifically, in one embodiment, novel asymmetric diamine compounds, including diamine compounds with asymmetrically fluorinated hydrocarbons, are used via condensation with tetra-carboxylic acid dianhydride monomer to give soluble aromatic polyimide with high solubility in an organic solvent and excellent processibility after imidization is complete.

Specifically, in another embodiment, provided is a diamine compound having two substituents introduced asymmetrically to inhibit several interactions from polyimide or polyamide, with the substituent being a bulky group.

The bulky group can be an electron withdrawing group, such as a linear or branched perfluorinated alkyl group. In another embodiment, the linear or branched perfluorinated alkyl group may be a trifluoromethyl group.

Also provided herein, in another embodiment, are polymers containing imide and/or amide synthesized from diamine compounds with two functional groups asymmetrically substituted. More specifically, in yet another embodiment, provided is a method of preparing polyamic acid and/or polyimide using diamine compounds with two functional groups asymmetrically substituted, and polyamic acid and/or polyimide thereof.

In still another embodiment, provided is a polyimide with excellent processibility and high solubility in an organic solvent after complete imidization using diamine compounds asymmetrically di-substituted with two functional groups. When polymerizing the diamine compounds asymmetrically substituted with two functional groups with tetracarboxylic acid dianhydride monomers, a fully aromatic polyimide is prepared with excellent solubility in organic solvents after the imidization is complete, superior processibility, and excellent thermal and optical properties.

In yet still another embodiment, provided is a soluble polyimide with high processibility and solubility in an organic solvent after complete imidization using diamine compounds asymmetrically substituted with two functional groups, such as fluorinated alkyl groups, by controlling molecular structure to inhibit several interactions between polyimide chains.

In one embodiment, provided is a thermoplastic polyimide which maintains the superior properties of polyimide such as robustness with rigid structure, and has high solubility after the final condensation state with high processibility and low refractive index. The properties are attributed to the unusual structure of the diamine compounds. In another embodiment, a film derived from the polyimide has excellent thermal, mechanical, and optical properties, which are useful in applications, such as electrical, electronic, or optical materials, as well as having high commercial value.

### BRIEF DESCRIPTION OF THE DRAWINGS

FIG. 1 is a ¹H NMR spectrum of a representative diamine compound from the preparation example.

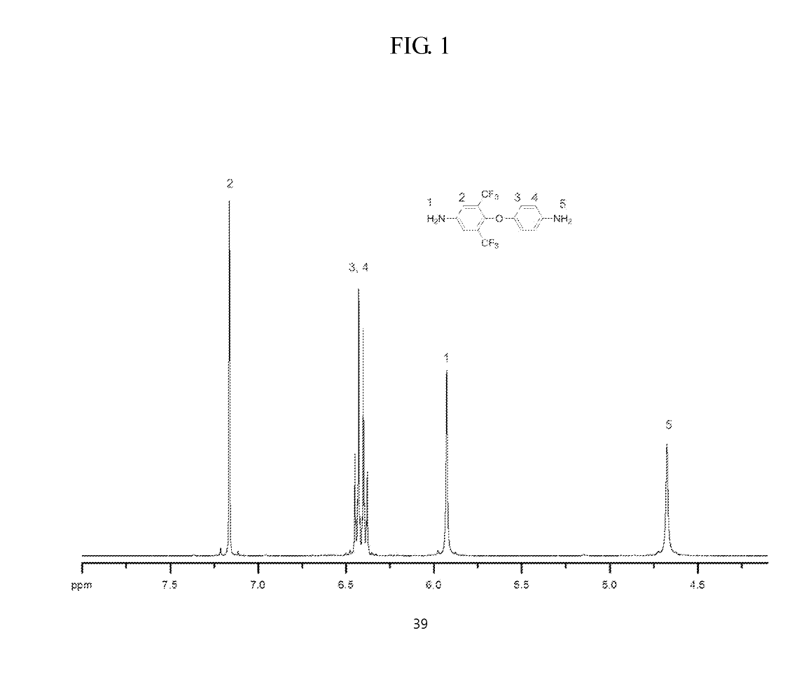

FIG. 2 is a ¹³C NMR spectrum of a representative diamine compound from the preparation example.

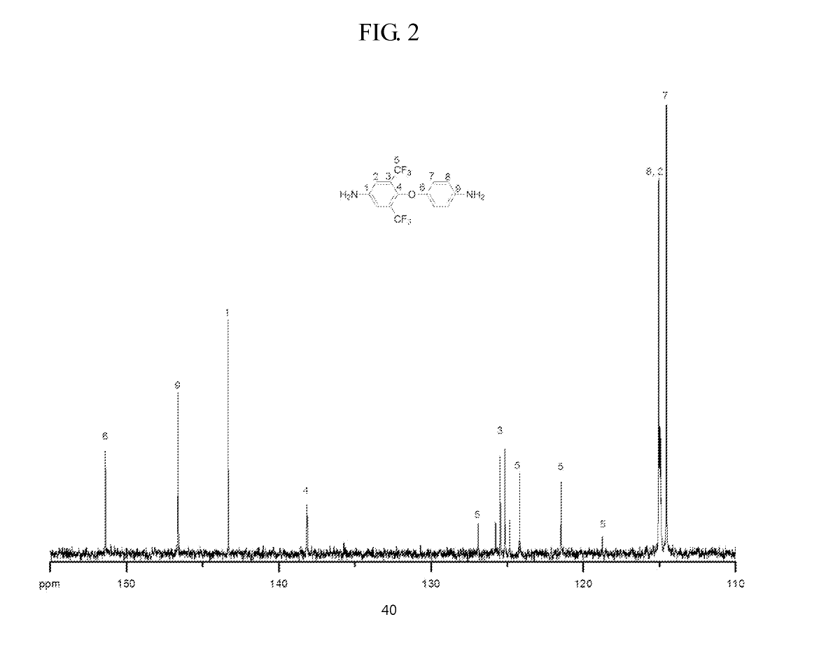

FIG. 3 is a ¹H NMR spectrum of a polyimide from Example 1.

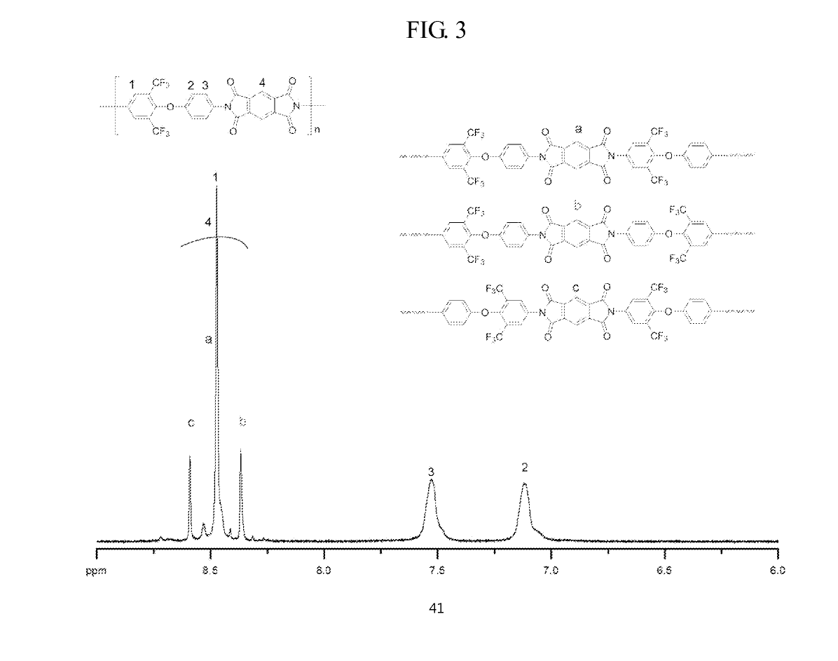

FIG. 4 is a ¹H NMR spectrum of a polyimide from Example 2.

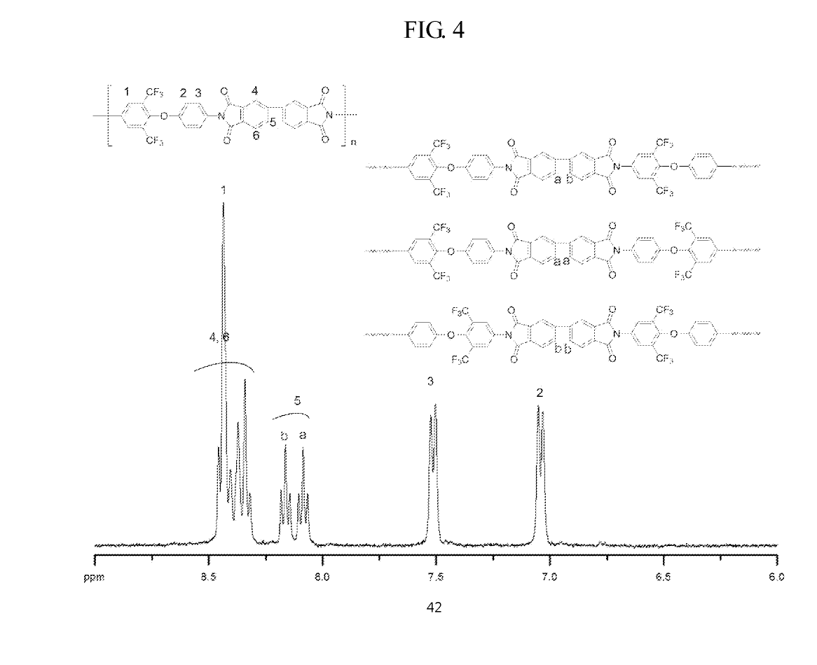

FIG. 5 is a ¹H NMR spectrum of a polyimide from Example 3.

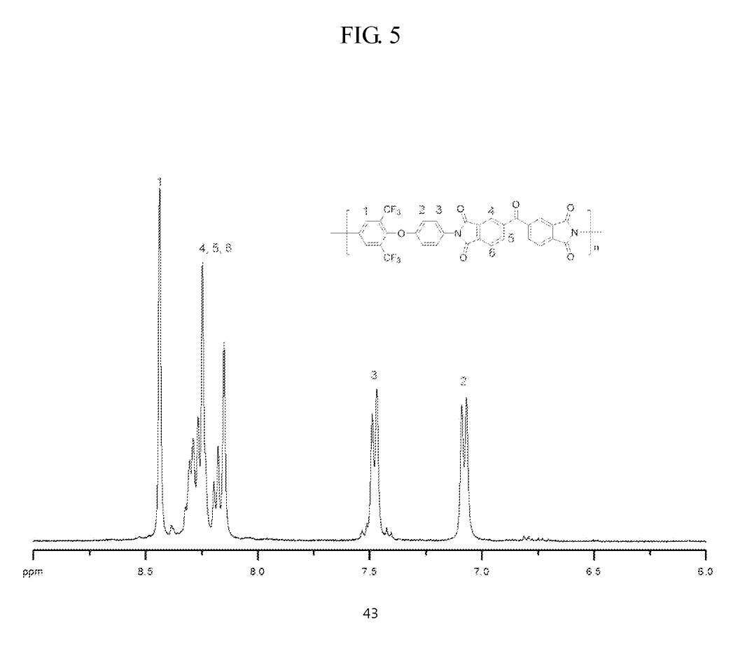

FIG. 6 is a ¹H NMR spectrum of a polyimide from Example 4.

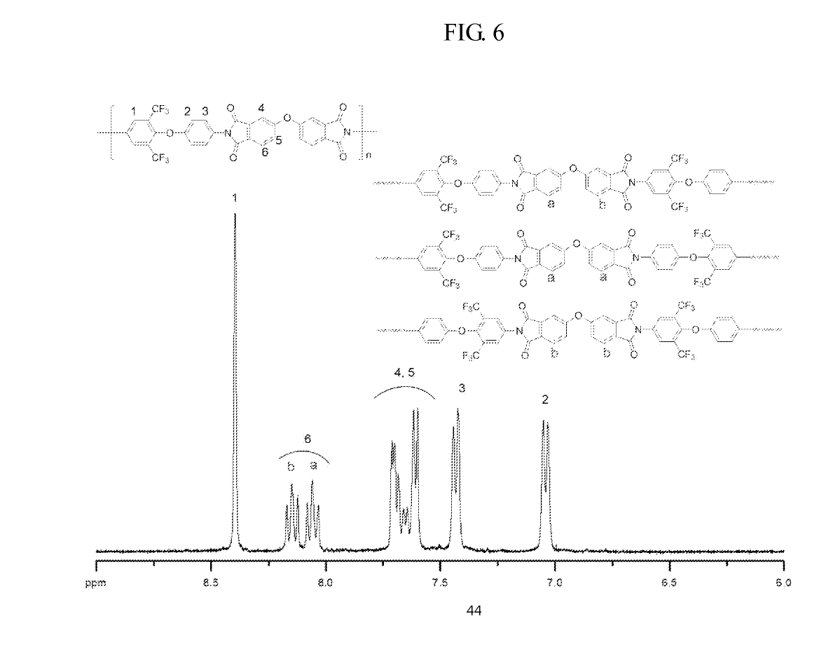

FIG. 7 is a ¹H NMR spectrum of a polyimide from Example 5.

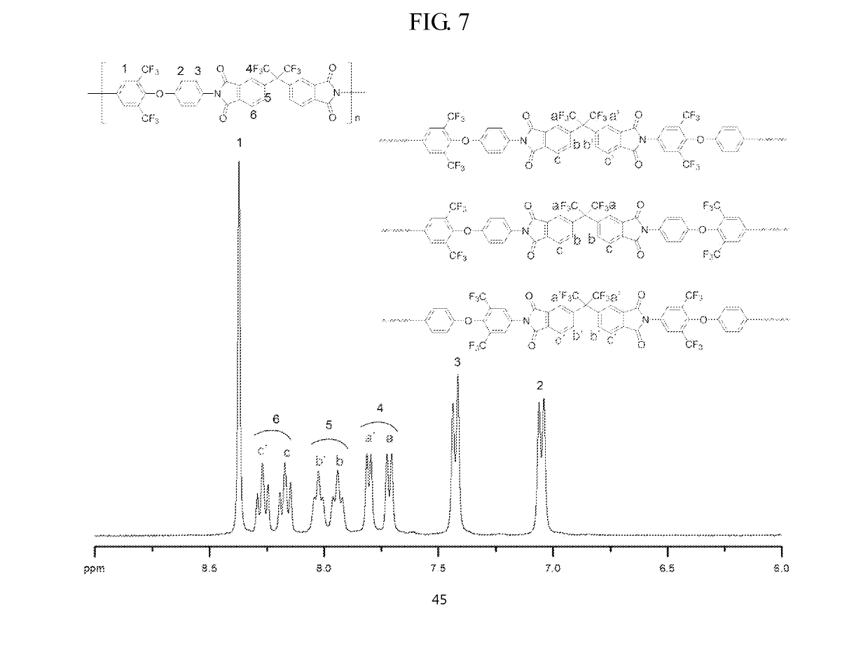

FIG. 8 is infrared (IR) spectra of polyimides from examples 1-5.

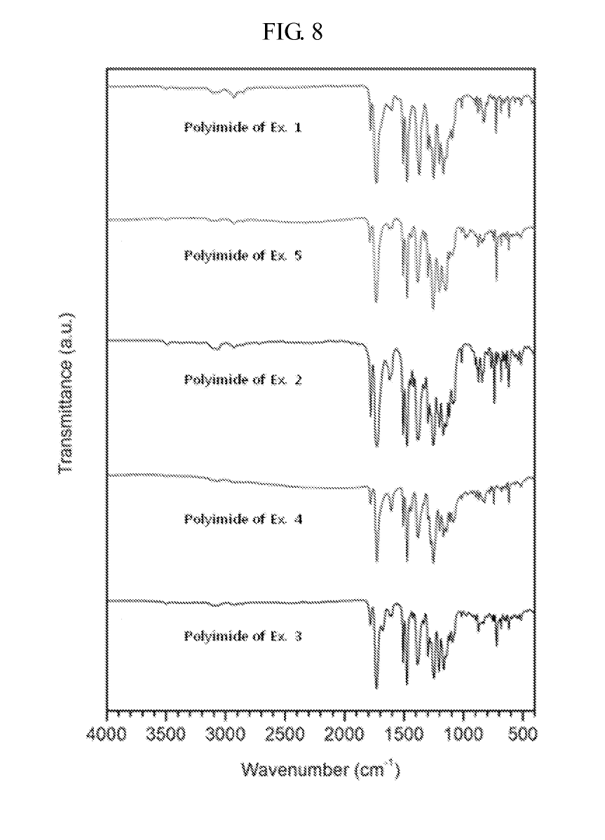

FIG. 9 is a DSC graph of polyimides from examples 1-5.

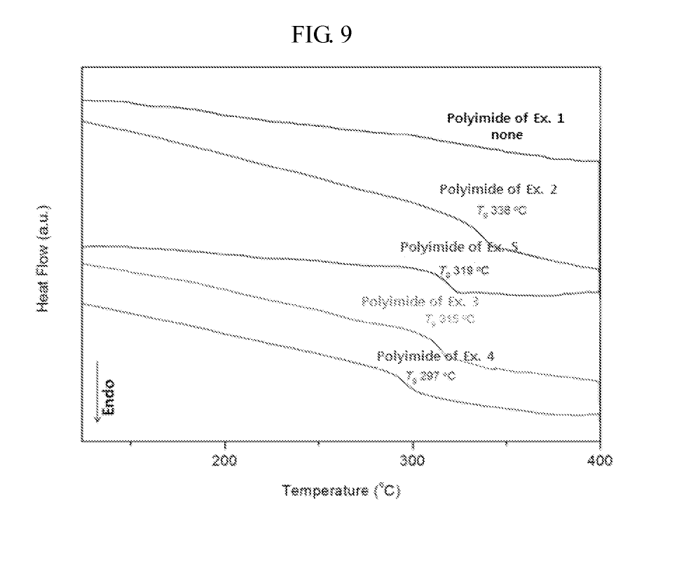

### DETAILED DESCRIPTION

Provide is an asymmetrical diamine compound having two substituents, R and R′ attached to cyclic group A of the asymmetrical compound as represented by Formula 1:

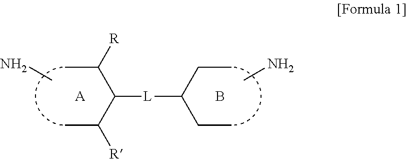

wherein R and R′ each independently represents (i) C1 to C12 alkyl group unsubstituted or substituted with one or more halogen, (ii) C1 to C12 alkoxy group unsubstituted or substituted with one or more halogen, (iii) C2 to C12 alkenyl group unsubstituted or substituted with one or more halogen, (iv) C2 to C12 alkynyl group unsubstituted or substituted with one or more halogen, (v) C4 to C30 cycloalkyl group unsubstituted or substituted with one or more halogen, (vi) cycloalkenyl group unsubstituted or substituted with one or more halogen, (vii) C6 to C30 aryl group unsubstituted or substituted with one or more halogen, C1 to C12 alkyl group, C1 to C12 alkoxy group, C1 to C12 halogenated alkyl group and/or C1 to C12 halogenated alkoxy group, (viii) C3 to C30 heteroaryl group unsubstituted or substituted with one or more halogen, C1 to C12 alkyl group, C1 to C12 alkoxy group, C1 to C12 halogenated alkyl group and/or C1 to C12 halogenated alkoxy group, or (ix) C6 to C30 arylalkyl group unsubstituted or substituted with one or more halogen, C1 to C12 alkyl group, C1 to C12 alkoxy group, C1 to C12 halogenated alkyl group and/or C1 to C12 halogenated alkoxy group;

The linker, ‘-L-’ represents a direct bond, —O—, —S—, —C(═O)O—, —OC(═O)—, —C(═O)—, —SO2-, C(CH3)2-, —C(CF3)2-, —NR″″—, or a combination thereof (where R″″ is hydrogen or C1 to C6 alkyl group unsubstituted or substituted with one or more halogen), and;

cyclic groups

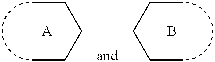

and each independently represent (i) C5 to C30 aryl or cycloalkyl group having at least one 5-membered or 6-membered ring unsubstituted or substituted with halogen, or (ii) C3 to C30 heteroaryl, or heterocycloalkyl group having at least one 5-membered or 6-membered ring unsubstituted or substituted with halogen.

In one embodiment, the substituents R and R′ may represent hydrocarbyl groups having fluorine, and/or trifluoromethyl groups.

Additionally, ‘-L’- may be any one selected from the group consisting of a direct bond, —O—, and —S—.

Additionally, in one embodiment, the asymmetric diamine is represented in Formula 2, wherein two trifluoromethyl groups are attached to one of the cyclic groups, asymmetrically.

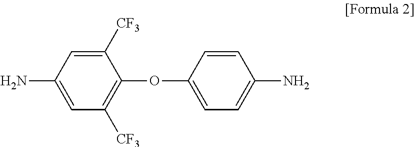 [SMILES: Nc1ccc(Oc2c(C(F)(F)F)cc(N)cc2C(F)(F)F)cc1]

In another embodiment, provided is a process as represented in Schemes 1 and 2, for producing an asymmetric diamine compound as represented in Formula 1, including synthesizing a dinitro compound as represented by Formula C (Scheme 1) via undergoing a nucleophilic substitution reaction between compounds represented as Formula A and Formula B (Scheme 1); followed by the hydrogenation of the dinitro compound (Formula C):

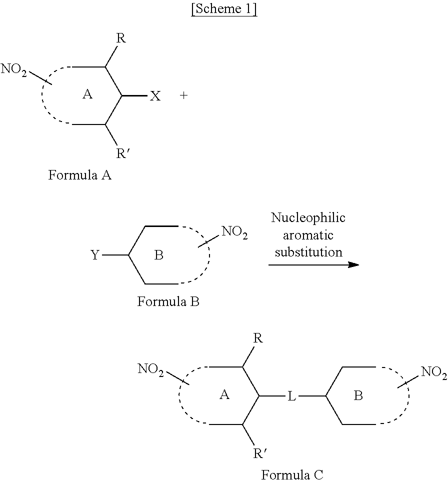

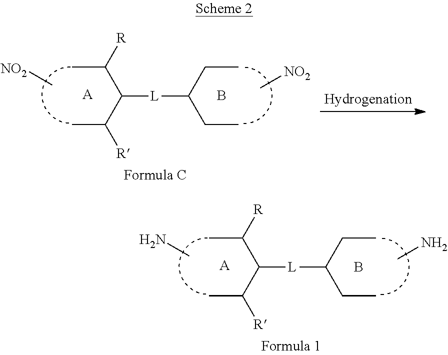

i) wherein, as in Scheme 1, one of the —X or —Y represents a halogen atom (e.g., fluorine (—F), chlorine (—Cl), bromine (—Br), or iodine (—I)), an ester group, an acid chloride (—C(O)Cl), or a sulfonyl chloride, while the other is a hydroxyl (—OH), thiol (—SH), or alkali metal salt thereof which forms the linker (-L-) in the dinitro Formula C as in Scheme 1.

ii) wherein R and R′ each independently represents (i) C1 to C12 alkyl group unsubstituted or substituted with one or more halogen, (ii) C1 to C12 alkoxy group unsubstituted or substituted with one or more halogen, (iii) C2 to C12 alkenyl group unsubstituted or substituted with one or more halogen, (iv) C2 to C12 alkynyl group unsubstituted or substituted with one or more halogen, (v) C4 to C30 cycloalkyl group unsubstituted or substituted with one or more halogen, (vi) cycloalkenyl group unsubstituted or substituted with one or more halogen, (vii) C6 to C30 aryl group unsubstituted or substituted with one or more halogen, C1 to C12 alkyl group, C1 to C12 alkoxy group, C1 to C12 halogenated alkyl group and/or C1 to C12 halogenated alkoxy group, (viii) C3 to C30 heteroaryl group unsubstituted or substituted with one or more halogen, C1 to C12 alkyl group, C1 to C12 alkoxy group, C1 to C12 halogenated alkyl group and/or C1 to C12 halogenated alkoxy group, or (ix) C6 to C30 arylalkyl group unsubstituted or substituted with one or more halogen, C1 to C12 alkyl group, C1 to C12 alkoxy group, C1 to C12 halogenated alkyl group and/or C1 to C12 halogenated alkoxy group;

iii) the linker ‘-L-’ represents a direct bond, —O—, —S—, —C(═O)O—, —OC(═O)—, or —SO₂—, and

the cyclic group

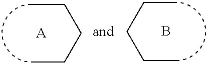

and K each independently represent (i) C5 to C30 aryl or cycloalkyl group having at least one 5-membered or 6-membered ring unsubstituted or substituted with halogen, or (ii) C3 to C30 heteroaryl, or heterocycloalkyl group having at least one 5-membered or 6-membered ring unsubstituted or substituted with halogen.

In one embodiment of the process for producing an asymmetric diamine, said substituents R and R′ may represent a hydrocarbyl group substituted with fluorine, and/or trifluoromethyl groups. Additionally, said ‘-L-’ may be any one selected from the group consisting of a direct bond, —O—, or —S—.

Additionally, the preparation method of diamine compound of the present invention provides with the preparation method of diamine compounds with two substituents attached asymmetrically described in Scheme 3.

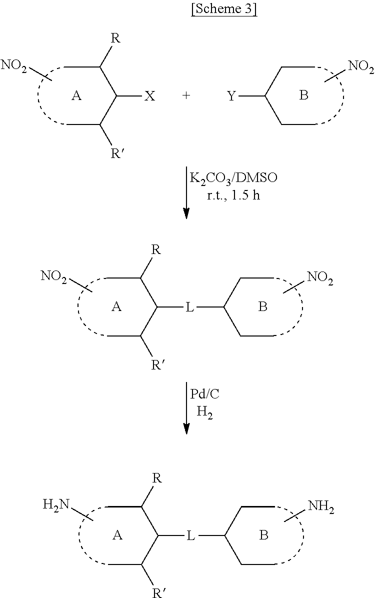

As shown in Scheme 3, one of the —X or —Y may represent a halogen atom (-hal) such as —F, —Cl, —Br, or —I, while the other may be a reactive functional group to form the linker -L- as in Formula 1 of the present invention. For example, if —X is -hal and —Y is —OH or SH, then the linker -L- is —O— or —S—, respectively, and the rest of the linkers (supra) may be easily selected from the linkers in Scheme 1 and Formula 1.

In one embodiment, examples of R and R′ may be independently selected from (i) C1 to C12 alkyl groups unsubstituted or substituted with one or more halogen, where C1 to C12 are linear or branched alkyl groups, such as methyl, ethyl, n-propyl, i-propyl, n-butyl, i-butyl, or t-butyl, optionally substituted with one or more halogen (i.e., fluoride), such as trifluoromethyl groups for R or R′.

In another embodiment, examples of R and R′ may be independently selected from (ii) C1 to C12 alkoxy groups unsubstituted or substituted with one or more halogen, where C1 to C12 are linear or branched alkoxy groups, such as methoxy, ethoxy, propoxy, or butoxy, optionally substituted with one or more halogen (i.e., fluoride), such as trifluoromethoxy groups for R or R′.

In yet another embodiment, examples of R and R′ may be independently selected from (iii) C2 to C12 alkenyl groups unsubstituted or substituted with one or more halogen, where C1 to C12 are linear or branched alkenyl groups, such as ethenyl, propenyl, or butenyl, optionally substituted with one or more halogen (i.e., fluoride), such as fluoroethenyl, fluoropropenyl, fluorobutenyl, trifluoromethylethenyl groups, and the like for R or R′.

In yet still another embodiment, examples of R and R′ may be independently selected from (iv) C2 to C12 alkynyl groups unsubstituted or substituted with one or more halogen, wherein C2 to C12 are linear or branched alkynyl groups, such as ethynyl, propynyl, or butynyl, optionally substituted with one or more halogen (i.e., fluoride), such as fluoroethynyl, fluoropropynyl, fluorobutynyl, trifluoromethylethynyl groups, and the like for R or R′.

In one embodiment, examples of R and R′ may be independently selected from (v) C4 to C30 cycloalkyl groups unsubstituted or substituted with one or more halogen, where C4 to C30 are linear or branched cycloalkyl groups, such as cyclobutyl, cyclopentyl, cyclohexyl, and the like, optionally substituted with one or more halogen (i.e., fluoride), such as fluorocyclobutyl, fluorocyclopentyl, fluorocyclohexyl, and the like for R or R′.

In another embodiment, examples of R and R′ may be independently selected from (vi) cycloalkenyl groups unsubstituted or substituted with one or more halogen, which are linear or branched cycloalkenyl groups, such as cyclopropenyl, cyclobutenyl, cyclopentenyl, cyclohexenyl, and the like, optionally substituted with one or more halogen (i.e., fluoride), such as fluorocyclobutenyl, fluorocyclopentenyl, fluorocyclohexenyl, and the like for R or R′.

In another embodiment, examples of R and R′ may be independently selected from (vii) C6 to C30 aryl groups unsubstituted or substituted with one or more halogen, and/or C1 to C12 alkyl, phenyl, naphthyl or aryl groups substituted with linear or branched alkyl groups or halogen, and the like.

In yet another embodiment, examples of R and R′ may be independently selected from (viii) C3 to C30 heteroaryl groups unsubstituted or substituted with one or more halogen, and/or C1 to C12 alkyl groups, where heteroaryl groups, such as pyrrole, pyridyl, thiophenyl, indolyl, etc., are optionally substituted with one or more halogen or C1 to C12 linear or branched alkyl groups.

In yet still another embodiment, examples of R and R′ may be independently selected from (ix) C6 to C30 arylalkyl groups unsubstituted or substituted with one or more halogen, and/or C1 to C12 alkyl groups, where arylalkyl groups are tolyl, mesityl, xylyl, etc., optionally substituted with one or more halogen or C1 to C12 linear or branched alkyl groups.

The substituting group of R and R′ may be a fluoroalkyl, fluoroalkoxy, or other substituted or unsubstituted aryl, or may be a perfluoroalkyl, perfluoroalkoxy, or other substituted or unsubstituted phenyl group, or may be trifluoromethyl (—CF3).

On the other hand, linker ‘-L-’ may be a direct bond, —O—, —S—, —NH—.

Also, in the preparation method of the diamine compounds, in the cases of cyclic groups A and B as 6-membered rings, the positions of amine groups may be para- to the linker ‘-L-’.

In one embodiment, the diamine compound of the present invention, obtained from the preparation method, is 2,6-bis(trifluoromethyl)-4,4′-diaminodiphenyl ether, represented as in Formula 2.

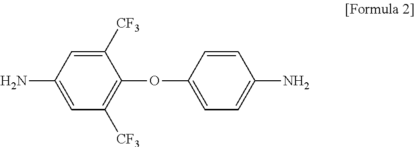 [SMILES: Nc1ccc(Oc2c(C(F)(F)F)cc(N)cc2C(F)(F)F)cc1]

More specifically, the diamine compound of Formula 2 may be prepared via the method represented as in Scheme 4, wherein -hal is —Br.

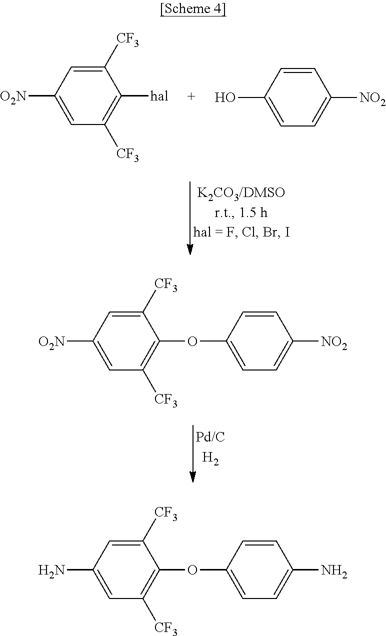

According to Scheme 2, the diamine compound of Formula 1 is obtained via treating 1-bromo-4-nitro-2,6-bis(trifluoromethyl)benzene with 4-nitrophenol in the presence of a base, such as calcium carbonate, to give the dinitro compound represented as Formula 3, followed by reduction of the nitro groups.

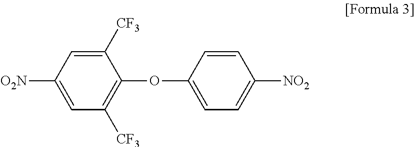 [SMILES: O=[N+]([O-])c1ccc(Oc2c(C(F)(F)F)cc([N+](=O)[O-])cc2C(F)(F)F)cc1]

Also, in one embodiment, provided is a process for producing a polymer selected from a group of polyamides, polyamic acids, and polyimides using the asymmetric diamine compound as a monomer.

More specifically, in one embodiment, provided is a process for producing polyimides represented as Formula 5 using the asymmetric diamine compound and tetracarboxylic acid as monomers in the polymerization reaction to give polyimides.

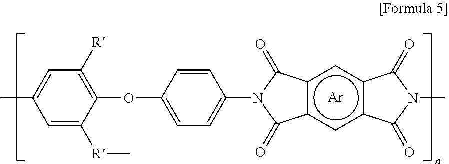

In formula 5, n is an integer selected from 1 to 10,000;

Ar is an optionally substituted aromatic group, with substituents selected from a group consisting of C1 to C20 alkyl groups, or C6 to C20 aryl groups, or optionally substituted heterocyclic aromatic groups, substituents selected from a group consisting of C1 to C20 alkyl groups, or C6 to C20 aryl groups; and

wherein R and R′ are the same as defined as above. The present invention is also related to a process of producing a polyimide comprising: solvating asymmetric diamine compound and tetracarboxylic acid in organic solvent as monomer to give polyamic acid, followed by heating, stirring to complete imidization reaction.

The tetracarboxylic acid is selected from a group consisting of linear hydrocarbon, cyclic hydrocarbon or aromatic hydrocarbon consisting of 4 carboxylic substitutents in a molecule, for example, aliphatic tetracarboxylic dianhydrides or alicyclic tetracarboxylic dianhydrides, such as butanetetracarboxylic dianhydride, cyclobutane-1,2,3,4-tetracarboxylic dianhydride, 1,2-dimethylcyclobutane-1,2,3,4-tetracarboxylic dianhydride, 1,3-Dimethylcyclobutane-1,2,3,4-tetracarboxylic dianhydride, 1,3-Dichlorocyclobutane-1,2,3,4-tetracarboxylic dianhydride, 1,2,3,4-Tetramethyl-1,2,3,4-tetracarboxylic dianhydride, Cyclopentane-1,2,3,4-tetracarboxylic dianhydride, Cyclohexane-1,2,4,5-tetracarboxylic dianhydride, Dicyclohexyl-3,3′4,4′-tetracarboxylic dianhydride, 2,3,5-Tricarboxycyclopentyl acetic dianhydride, 3,5,6-Tricarboxynorbonane-2-acetic dianhydride, Tetrahydrofuran-2,3,4,5-tetracarboxylic dianhydride, 1,3,3a,4,5,9b-Hexahydro-5-(tetrahydro-2,5-dioxo-3-furanyl)-naphtho[1,2-C]furan-1,3-dione, 1,3,3a,4,5,9b-Hexahydro-5-methyl-5-(tetrahydro-2,5-dioxo-3-furanyl)-naphtho[1,2-c]-furan-1,3-dione, 1,3,3a,4,5,9b-Hexahydro-5-ethyl-5-(tetrahydro-2,5-dioxo-3-furanyl)-naphtho[1,2-c]-furan-1,3-dione, 1,3,3a,4,5,9b-Hexahydro-7-methyl-5-(tetrahydro-2,5-dioxo-3-furanyl)-naphtho[1,2-c]-furan-1,3,-dione, 1,3,3a,4,5,9b-Hexahydro-7-ethyl-5-(tetrahydro-2,5-dioxo-3-furanyl)-naphtho[1,2-c]-furan-1,3,-dione, 1,3,3a,4,5,9b-Hexahydro-8-methyl-5-(tetrahydro-2,5-dioxo-3-furanyl)-naphtho[1,2-c]-furan-1,3-dione, 1,3,3a,4,5,9b-Hexahydro-8-ethyl-5-(tetrahydro-2,5-dioxo-3-furanyl)-naphtho[1,2-c]-furan-1,3-dione, 1,3,3a,4,5,9b-Hexahydro-5,8-dimethyl-5-(tetrahydro-2,5-dioxo-3-furanyl)-naphtho[1,2-c]-furan-1,3-dione, 5-(2,5-Dioxotetrahydrofuranyl)-3-methyl-3-cyclohexene-1,2-dicarboxylic dianhydride, Bicyclo[2.2.2]oct-7-ene-2,3,5,6-tetracarboxylic dianhydride, 3-Oxabicyclo[3.2.1]octane-2,4-dione-6-spiro-3′-(tetrahydrofuran-2′,5′-dione), 5-(2,5-Dioxotetrahydro-3-furanyl)-3-methyl-3-cyclohexene-1,2-dicarboxylic anhydride, 3,5,6-Tricarboxy norbornane-2-acetic dianhydride, and 4,9-Dioxatricyclo[5.3.1.02,6]undecane-3,5,8,10-tetraone, without limitation.

Additionally, the tetracarboxylic acid is selected from aromatic tetracarboxylic dianhydrides, such as pyromellitic dianhydride (1,2,4,5-benzenetetracarboxylic anhydride), 3,3′,4,4′-biphenyltetracarboxylic dianhydride, 2,2′,3,3′-biphenyltetracarboxylic dianhydride, 3,3′,4,4′-benzophenonetetracarboxylic dianhydride, 2,2′,3,3′-benzophenonetetracarboxylic dianhydride, 1,4,5,8-naphthalenetetracarboxylic dianhydride, 1,2,5,6-naphthalenetetracarboxylic dianhydride, 2,3,6,7-naphthalenetetracarboxylic dianhydride, 3,3′,4,4′-biphenyl ether tetracarboxylic acid dianhydride, 2,2′,3,3′-biphenyl ether tetracarboxylic acid dianhydride, 3,3′,4,4′-tetracarboxydiphenylsulfide dianhydride, 2,2′,3,3′-tetracarboxydiphenylsulfide dianhydride, 3,3′,4,4′-tetracarboxydiphenylsulfone dianhydride, 2,2′,3,3′-tetracarboxydiphenylsulfone dianhydride, 3,3′,4,4′-perfluoroisopropylidenediphthalic dianhydride, 3,3′,4,4′-dimethyldiphenylsilanetetracarboxylic dianhydride, 3,3′,4,4′-tetraphenylsilanetetracarboxylic dianhydride, 1,2,3,4-furantetracarboxylic dianhydride, 4,4′-bis(3,4-dicarboxyphenoxy)diphenyl sulfide dianhydride, 4,4′-bis(3,4-dicarboxyphenoxy)diphenyl sulfone dianhydride, 4,4′-bis(3,4-dicarboxyphenoxy)diphenyl propane dianhydride, Bis(phthalic acid)phenylphosphine oxide dianhydride, p-phenylene-bis(triphenylphthalic)dianhydride, m-phenylene-bis(triphenylphthalic)dianhydride, Bis(triphenylphthalic acid)-4-4′-diphenylether dianhydride, Bis(triphenylphthalic acid)-4-4′-diphenylmethane dianhydride, Ethyleneglycol bis(anhydrotrimelitate), Propyleneglycol bis(anhydrotrimelitate), 1,4-butanediol bis(anhydrotrimelitate), 1,6-hexanediol bis(anhydrotrimelitate), 1,8-oxtanediol bis(anhydrotrimelitate), and 2,2-bis(4-hydroxyphenyl)propane bis(anhydrotrimelitate), without limitation.

The tetracarboxylic dianhydrides can be used, either alone or as a mixture of two or more, selected from the group consisting of 1,2,4,5-Benzenetetracarboxylic dianhydride, 3,3′,4,4′-Benzophenonetetracarboxylic dianhydride, 2,2′,3,3′-Benzophenonetetracarboxylic dianhydride, 3,3′,4,4′-Diphenylethercarboxylic dianhydride, 2,2′,3,3′-Diphenylethercarboxylic dianhydride, 3,3′-Oxydiphthalic dianhydride, 3,3′,4,4′-Biphenyltetracarboxylic dianhydride, 2,2′,3,3′-Biphenyltetracarboxylic dianhydride, Diphenyl sulfide-3,3′,4,4′-tetracarboxylic dianhydride, Diphenyl sulfide-2,2′,3,3′-tetracarboxylic dianhydride, Diphenyl sulfone-3,3′,4,4′-tetracarboxylic dianhydride, Diphenyl sulfone-2,2′,3,3′-tetracarboxylic dianhydride, 4,4′-(hexafluoroisopropylidene)diphthalic anhydride, 1,4,5,8-naphthalenetetracarboxylic dianhydride, 1,2,5,6-naphthalenetetracarboxylic dianhydride, and 2,3,6,7-naphthalenetetracarboxylic dianhydride, without limitation.

Also, in the preparation method of polyimide, another diamine compound can be mixed with the asymmetric diamine compounds of the present invention, depending on the purpose or usage of the polymer, so as long as not changing the properties of the polymer, such as solubility and transparency.

The additional diamine compound is selected from a group consisting of linear hydrocarbon, cyclic hydrocarbon, and aromatic hydrocarbon consisting of two amine groups within one molecule, for example, aromatic diamine, such as p-Phenylenediamine, m-Phenylenediamine, p-Aminobenzylamine, m-Aminobenzylamine, 4,4′-Diaminodiphenylmethane, 3,4′-Diaminodiphenylmethane, 3,3′-Diaminodiphenylmethane, 4,4′-Diaminodiphenylethane, 4,4′-Diaminobenzanilide, 4,4′-Diaminodiphenyl ether, 3,4′-Diaminodiphenyl ether, 3,3′-Diaminodiphenyl ether, 2,4′-Diaminodiphenyl ether, 2,2′-Diaminodiphenyl ether, 2,3′-Diaminodiphenyl ether, 1,4-Bis(4-aminophenoxy)benzene, 1,4-Bis(3-aminophenoxy)benzene, 1,3-Bis(4-aminophenoxy)benzene, 1,3-Bis(3-aminophenoxy)benzene, 2,2-Bis[4-(4-aminophenoxyl)phenyl]propane, 2,2-Bis[4-(3-aminophenoxyl)phenyl]propane, Bis(4-aminophenyl)sulfide, Bis(3-aminophenyl)sulfide, 3,4-Diaminophenyl sulfide, Bis(4-aminophenyl)sulfoxide, Bis(3-aminophenyl)sulfoxide, 3,4-Diaminophenyl sulfoxide, Bis(4-aminophenyl)sulfone, Bis(3-aminophenyl)sulfone, 3,4-Diaminophenyl sulfone, 4,4′-Diaminobenzophenone, 3,4′-Diaminobenzophenone, 3,3′-Diaminobenzophenone, Bis[4-(4-aminophenoxyl)phenyl]sulfone, Bis[4-(3-aminophenoxyl)phenyl]sulfone, Bis[4-(4-aminophenoxyl)phenyl]ether, 1,4-Bis[4-(3-aminophenoxyl)benzoyl]benzene, 1,3-Bis[4-(3-aminophenoxyl)benzoyl]benzene, 4,4′-Bis[3-(4-aminophenoxyl)benzoyl]diphenyl ether, 4,4′-Bis[3-(3-aminophenoxyl)benzoyl]diphenyl ether, 4,4′-Bis[4-(4-amino-α,α-dimethylbenzyl)phenoxy]benzophenone, 4,4′-Bis[4-(4-amino-α,α-dimethylbenzyl)phenoxy]diphenyl sulfone, Bis[4-{4-(4-aminophenoxyl)phenoxy}phenyl]sulfone, 1,4-Bis[4-(4-aminophenoxy)-α,α-dimethylbenzyl]benzene, 1,3-Bis[4-(4-aminophenoxy)-α,α-dimethylbenzyl]benzene, 2,2-Bis[4-(4-aminophenoxyl)phenyl]-1,1,1,3,3,3-hexafluoropropane, 2,2-Bis[4-(3-aminophenoxyl)phenyl]-1,1,1,3,3,3-hexafluoropropane, 1,3-Diaminonaphthalene, 1,4-Diaminonaphthalene, 1,5-Diaminonaphthalene, 2,6-Diaminonaphthalene, 2,2′-Dimethyl-4,4′-diaminobiphenyl, 3,3′-Dimethyl-4,4′-diaminobiphenyl, 2,2′-Ditrifluoromethyl-4,4′-diaminobiphenyl, 3,3′-Ditrifluoromethyl-4,4′-diaminobiphenyl, 5-Amino-1-(4′-aminophenyl)-1,3,3-trimethylindane, 6-Amino-1-(4′-aminophenyl)-1,3,3-trimethylindane, 2,2-Bis[4-(4-aminophenoxyl)phenyl]propane, 2,2-Bis[4-(4-aminophenoxyl)phenyl]hexafluoropropane, 2,2-Bis(4-aminophenyl)hexafluoropropane, 2,2-Bis[4-(4-aminophenoxyl)phenyl]sulfone, 1,4-Bis(4-aminophenoxy)benzene, 1,3-Bis(4-aminophenoxy)benzene, 1,3-Bis(3-aminophenoxy)benzene, 9,9-Bis(4-aminophenyl)-10-hydroanthracene, 2,7-Diaminofluorene, 9,9-Dimethyl-2,7-diaminofluorene, 9,9-Bis(4-aminophenyl)fluorene, 4,4′-Methylene-Bis(2-chloroaniline), 2,2′,5,5′-Tetrachloro-4,4′-diaminobiphenyl, 2,2′-Dichloro-4,4′-diamino-5,5′-dimethoxybiphenyl, 3,3′-Dimethoxy-4,4′-diaminobiphenyl, 4,4′-(p-phenyleneisopropylidene)bisaniline, 4,4′-(m-phenyleneisopropylidene)bisaniline, 2,2′-Bis[4-(4-amino-2-trifluoromethylphenoxy)phenyl]hexafluoropropane, 4,4′-Diamino-2,2′-Bis(trifluoromethyl)biphenyl, and 4,4′-Bis[(4-amino-2-trifluoromethyl)phenoxy]-octafluorobiphenyl, without limitation.

Additionally, the diamine compound can be either an aliphatic diamine or an alicyclic diamine, such as 1,1-methaxylylenediamine, 1,3-propanediamine, Tetramethylenediamine, Pentamethylenediamine, Hexamethylenediamine, Heptamethylenediamine, Octamethylenediamine, Nonamethylenediamine, 1,4-Diaminocyclohexane, Isophoronediamine, Tetrahydrodicyclopentadienylenediamine, Hexahydro-4,7-methanoindanylenedimethylenediamine, Tricyclo[6,2,1,0².7]-undecyclenedimethyldiamine, and 4,4′-methylenebis(cyclohexylamine), without limitation.

Also, diamines having two primary amine and another nitrogen atoms other than the primary amine, such as 2,3-Diaminopyridine, 2,6-Diaminopyridine, 3,4-Diaminopyridine, 2,4-Diaminopyrimidine, 5,6-Diamino-2,3-dicyanopyrazine, 5,6-Diamino-2,4-dihydroxypyrimidine, 2,4-Diamino-6-dimethylamino-1,3,5-triazine, 1,4-Bis(3-aminopropyl)piperazine, 2,4-Diamino-6-isopropoxy-1,3,5-triazine, 2,4-Diamino-6-methoxy-1,3,5-triazine, 2,4-Diamino-6-phenyl-1,3,5-triazine, 2,4-Diamino-6-methyl-s-triazine, 2,4-Diamino-1,3,5-triazine, 4,6-Diamino-2-vinyl-s-triazine, 2,4-Diamino-5-phenylthiazole, 2,6-Diaminopurine, 5,6-Diamino-1,3-dimethyluracil, 3,5-Diamino-1,2,4-triazole, 6,9-Diamino-2-ethoxyacridine lactate, 3,8-Diamino-6-phenylphenanthridine, 1,4-Diaminopiperazine, 3,6-Diaminoacridine, Bis(4-aminophenyl)phenylamine, 3,6-Diaminocarbazole, N-Methyl-3,6-diaminocarbazole, N-Ethyl-3,6-diaminocarbazole, N-Phenyl-3,6-diaminocarbazole, N,N′-Di(4-aminophenyl)-benzidine can be also used along with the diamine compound represented as Formula 2, as well as diaminoorganosiloxane, diamine with steroid, and rigid diamine with acetylene, without limitation.

Additional diamines that can be used may be selected from a group consisting of 4,4′-Diaminodiphenyl ether, 3,4′-Diaminodiphenyl ether, 3,3′-Diaminodiphenyl ether, 2,4′-Diaminodiphenyl ether, 2,2′-Diaminodiphenyl ether, 2,3′-Diaminodiphenyl ether, 1,4-Bis(4-aminophenoxy)benzene, 1,4-Bis(3-aminophenoxy)benzene, 1,3-Bis(4-aminophenoxy)benzene, 1,3-Bis(3-aminophenoxy)benzene, p-Phenylenediamine, m-Phenylenediamine, o-Phenylenediamine, p-Aminobenzylamine, m-Aminobenzylamine, 4,4′-diaminodiphenylmethane, 3,4′-diaminodiphenylmethane, 3,3′-diaminodiphenylmethane, 2,2-Bis[4-(4-aminophenoxyl)phenyl]propane, 2,2-Bis[4-(3-aminophenoxyl)phenyl]propane, Bis(4-aminophenyl)sulfide, Bis(3-aminophenyl)sulfide, 3,4-diaminophenyl sulfide, Bis(4-aminophenyl)sulfoxide, Bis(3-aminophenyl)sulfoxide, 3,4-diaminophenyl sulfoxide, Bis(4-aminophenyl)sulfone, Bis(3-aminophenyl)sulfone, 3,4-diaminophenyl sulfone, 4,4′-diaminobenzophenone, 3,4′-diaminobenzophenone, 3,3′-diaminobenzophenone, Bis[4-(4-aminophenoxyl)phenyl)]sulfone, Bis[4-(3-aminophenoxyl)phenyl)]sulfone, Bis[4-(4-aminophenoxyl)phenyl)]ether, 1,4-Bis[4-(3-aminophenoxyl)benzoyl]benzene, 1,3-Bis[4-(3-aminophenoxyl)benzoyl]benzene, 4,4′-Bis[3-(4-aminophenoxyl)benzoyl]diphenyl ether, 4,4′-Bis[3-(3-aminophenoxyl)benzoyl]diphenyl ether, 4,4′-Bis[4-(4-amino-α,α-dimethylbenzyl)phenoxy]benzophenone, 4,4′-Bis[4-(4-amino-α,α-dimethylbenzyl)phenoxy]diphenyl sulfone, Bis[4-{4-(4-aminophenoxyl)phenoxy}phenyl]sulfone, 1,4-Bis[4-(4-aminophenoxy)-α,α-dimethylbenzyl]benzene, 1,3-Bis[4-(4-aminophenoxy)-α,α-dimethylbenzyl]benzene, 2,2-Bis[4-(4-aminophenoxyl)phenyl]-1,1,1,3,3,3-hexafluoropropane, and 2,2-Bis[4-(3-aminophenoxyl)phenyl]-1,1,1,3,3,3-hexafluoropropane, without limitation.

Also, in one embodiment, provided is a polymer selected from the group consisting of polyamide, polyamic acid, and polyimide, which result from the polymerization of asymmetric diamine as monomers.

More specifically, the polymer is synthesized by imidation of asymmetric diamine compound and tetra-carboxylic acid as monomers, with the resulting polyimide represented as Formula 5.

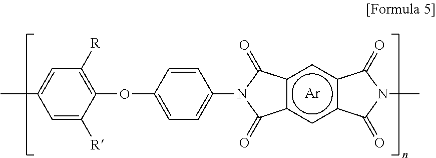

In formula 5, n is an integer selected from 1 to 10,000;

Ar is an optionally substituted aromatic group, with substituents selected from a group consisting of a C1 to C20 alkyl group, or a C6 to C20 aryl group, or optionally a substituted heterocyclic aromatic group, wherein the substituents may be selected from a group consisting of a C1 to C20 alkyl group, or a C6 to C20 aryl group; and

wherein R and R′ are the same as defined as above.

As described above, in the case of the introduction of asymmetric substituents, especially bulky, electron withdrawing groups, an aromatic polyimide resulting from polymerization with the asymmetric diamine, reduces the interactions between chains by cancelling symmetry, thus making the polyimide more soluble in organic solvents and more transparent in the form of films.

The tetra-carboxylic monomer used in the preparation of the polyimide are any one of the groups mentioned above, and the asymmetric diamine compound can be used as a mixture of another diamine to give a polyamic acid, followed by the formation of the polyimide in the imidization.

Also, in one embodiment, provided is a film that is prepared by dissolving the polyimide in a polar aprotic organic solvent or aromatic alcohol.

The polar aprotic organic solvent may be selected from a group consisting of N,N-dimethylformamide (DMF), N,N-dimethyl acetamide (DMAc), N-methyl pyrrolidone (NMP), dimethyl sulfoxide (DMSO), tetrahydrofuran (THF), and anisole, and the aromatic alcohol solvent is m-cresol.

In one embodiment, the polymer in the present invention is an aromatic polyamic acid represented as Formula 6.

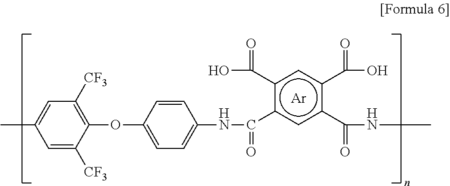

For example, the quaternary organic functional group represented as Ar of the aromatic ring is derived from the aromatic tetracarboxylic dianhydride.

In another embodiment, the polymer of the present invention is an aromatic polyimide represented as Formula 7, synthesized from the imidation of polyamic acid represented as Formula 6.

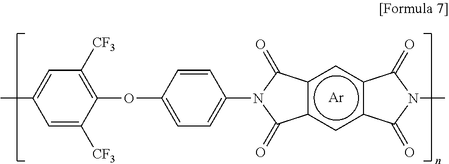

Specifically, in the preparation process of the polyamic acid and polyimide, equal portions of the asymmetric diamine compound of the present invention and the tetracarboxylic dianhydride are dissolved in a polar solvent, followed by stirring at room temperature to give the polyamic acid of Formula 6.

The reaction concentration may be 10-20% (i.e., weight of monomer (g)/amount of solvent (ml)). The concentration of the solution was diluted to a concentration of 5-10%, followed by raising the temperature while stirring to complete imidation. At this point, small amounts of dehydrating agent or imidation catalyst are added to more efficiently remove water formed during the imidation reaction. The dehydrating agent or imidization catalyst may be any dehydrating agent or imidization catalyst known to a person skilled in the art.

After the completion of the imidization reaction, the reaction mixture is added to an excess amount of a mixture of methanol and water to form precipitates, which are then washed with hot water and alcohol, followed by drying in a vacuum oven.

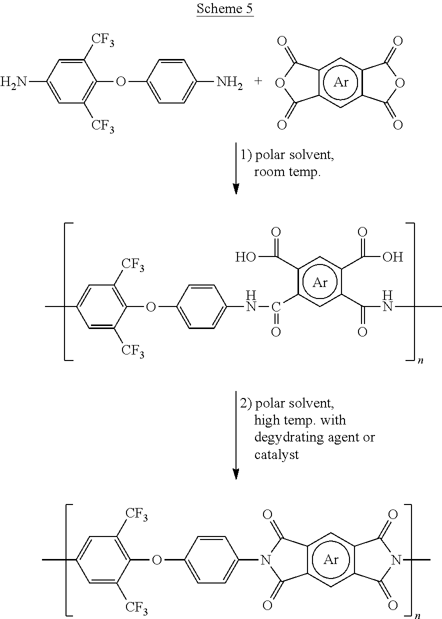

There are other methods for preparing polyimides via imidization of polyamic acids resulting in powders or films at 300 degrees using the asymmetric diamine compound of the present invention. However, said methods are difficult in that polyamic acid has poor storage stability, and side products, such as water are formed during the imidation process, thus making the processing of the polymer in the desired form difficult.

The diamine of the present invention may contain 0.1 mol % or more of the asymmetric diamine with respect to the whole diamine, or 20 mol % or more, or 50 mol % or more, or 80 mol % or more of the asymmetric diamine.

With the above-mentioned ratio, polymerizations using diamines including the diamine compound represented in Formula 2 gives a polymer having better heat resistance, transparency, and solubility.

### EXAMPLES

The diamines and polyimides of the present invention will be understood more clearly from the Examples outlined below, and are not meant to limit the scope of the invention. Simple modifications of the present invention may be accomplished by a person having ordinary skill in the art, and as such any of these modifications are included in the present invention.

The structure and properties of monomers and polymers in accordance with at least one embodiment are measured using the following methods.

The structure of the synthesized material was determined by IR (UV spectroscopy) and NMR. IR spectra was obtained from potassium bromide (KBR) or thin-film using a Bruker EQUINOX-55 spectrophotometer, and NMR spectra was obtained by dissolving compounds in chloroform, dimethyl sulfoxide-d6, then using a Bruker Fourier Transform AVANCE 400 spectrometer. The molecular weights of the synthesized polymers were measured at 35° C. by gel permeation chromatography (GPC) via dissolving the compound in tetrahydrofuran (THF).

The GPC was measured using a PLgel 10 μm MIXED-B column and a Viscotek TDA 302 refractive index detector. Thermogravimetric Analysis (TGA), Differential Scanning Calorimetry (DSC), and Thermomechanical Analysis (TMA) were measured using TA TGA Q500, DSC Q100, and TMA 2940 instruments, respectively.

TGA and DSC in the case of 10° C./min rate of increase of temperature was measured by, TMA in the case of 5° C./min rate of increase of temperature was measured. The thermal analysis, all measured under a constant nitrogen flow, TGA analysis was performed under a constant air flow.

Temperatures of 5% and 10% weight loss were measured from the TGA analysis, the glass transition temperature (T g) was chosen by selecting the middle part where there was a change in the slope of the curve, and the coefficient of thermal expansion (CTE) was measured using TMA in the temperature range between 50 and 250° C.

The refractive index was determined by a Sairon SPA-4000 prism coupler using a 630 and 1310 nm wavelength laser as the light source. Measurements were done at room temperature by preparing films with a thickness of 2-8 μm at room temperature in the horizontal and vertical directions.

### Examples of the Syntheses of Diamine Compounds

### Synthesis of 2,6-bis(trifluoroemthyl)-4,4′-diaminodiphenyl ether

Disodium phosphate 18.2 g (123 mmol) and tetrabutylammonium hydrogen sulfate 2.1 g (6.20 mmol) were dissolved in a 500 mL solution of acetone and dichloromethane, followed by the addition of 4-bromo-3,5-bis-trifluoromethyl aniline 10.0 g (32.5 mmol) dropwise with a oxone and the reaction solution was stirred for 1 hour at 0° C. Potassium hydroxide was added to maintain the acidity of the reaction solution between 7.5 and 8.5. After completion of the reaction, the solution was diluted with dichloromethane and washed with distilled water several times to remove salts. Magnesium sulfate was then added to the dichloromethane solution and the solvent was filtered then evaporated and the resulting reactant was passed through a silica column pale to give a light yellow compound, 1-bromo-4-nitro-2,6-bis(trifluoromethyl)benzene (8.05 g, 23.8 mmol 73.3% yield).

Melting point: 56-57° C.

¹H NMR (CDCl₃, 400 MHz, ppm): 8.71 (s, 2H).

¹³C NMR (DMSO-d₆, 100 MHz, ppm): 146.62, 132.48 (q, J=31.9 Hz), 126.71 (q, J=5.7 Hz), 125.63, 121.68 (q, J=272.9 Hz).

1-bromo-4-nitro-2,6-bis-trifluoromethyl benzene, 6.99 g (20.7 mmol) and 4-nitrophenol 3.16 g (22.7 mmol) were dissolved in 40 mL of dimethyl sulfoxide, then potassium carbonate (K2CO3) 4.29 g (31.0 mmol) was added and the mixture was stirred for 1.5 hours.

The reaction was diluted with 300 mL of ethyl acetate, followed by extraction with distilled water several times to remove the dimethyl sulfoxide and salts. To the ethyl acetate solution was added anhydrous magnesium sulfate to remove water, followed by passing the resulting reactant through a silica column to obtain a yellow dinitro compound, 2,6-bis-trifluoromethyl-4,4′-dinitro ether (8.20 g, 20.7 mmol; yield 100%).

¹H NMR (CDCl₃, 400 MHz, ppm): 8.831 (s, 2H), 8.181 (d, J=9.6 Hz, 2H), 6.891 (d, J=9.6 Hz, 2H).

¹³C NMR (CDCl₃, 100 MHz, ppm): 162.48, 153.74, 144.91, 143.74, 128.17 (q, J=34.3 Hz), 127.39 (q, J=5.0 Hz), 125.87, 121.02 (q, J=274.8 Hz), 115.95.

The dinitro compound 8 g (20.2 mmol) and 5% palladium on carbon 4 g were poured onto a mixture of 160 mL of ethyl acetate and 160 mL of ethanol, and stirred under hydrogen gas for three days. After the reaction, palladium-carbon was removed using a filter, followed by the evaporation of ethanol and ethyl acetate to yield a yellow diamine compound. The compound was passed through silica column and the resulting product was then recrystallized in a mixture of chloroform and hexane, followed by sublimation at 130° C. by vacuum sublimation to get white crystals of 2,6-bis-trifluoromethyl-4,4′-amino-diphenylether (6.6 g, 19.6 mmol; yield: 97%).

Melting point: 138-139° C.

¹H NMR (DMSO-d₆, 400 MHz, ppm): 7.164 (s, 2H), 6.414 (m, 4H), 5.927 (s, 2H), 4.675 (s, 2H).

¹³C NMR (DMSO-d₆, 100 MHz, ppm): 151.38, 146.61, 143.32, 138.15, 125.31 (q, J=30.7 Hz), 122.83 (q, J=273.7 Hz), 115.06, 115.00, 114.56.

FTIR (KBr, cm⁻¹):

### Example 1

### Preparation of Soluble Polyimide

Diamine compounds 0.39801 g (1.184 mmol) prepared (supra) were completely dissolved in 4.9 mL of purified NMP and an equivalent amount of 1,2,4,5-benzene tetracarboxylic dianhydride (PMDA) 0.25850 g (1.185 mmol) as a solid was added to the solution at room temperature and the solution was stirred for 4 hours to give polyamic acid.

To the solution was added 4.9 mL of NMP solvent, and the temperature was raised to 190° C., and a small amount of chlorobenzene was added to remove water produced during imidization and the solution was stirred for 6 hours. In this case, precipitation or gel formation during the reaction was not observed. After cooling to room temperature by diluting with 2 ml of NMP, the viscous solution was treated with a mixture of methanol and water to promote precipitation, which was washed several times with excess amounts of water and hot methanol, followed by drying in vacuum at 70° C. to obtain polymer.

1H NMR (DMSO-d₆, 400 MHz, ppm): 8.479 (m, 2H), 8.473 (s, 2H), 7.526 (m, 2H), 7.118 (m, 2H).

Some of the synthesized polymer was made as a 7.5% solution of DMAc by weight that was coated onto glass plate, followed by placing the glass plate at 100° C. under vacuum for 24 hours to remove the solvent to obtain a transparent and rigid film.

FTIR (film, cm-1): 1728, 1732 (C═O stretching of imide); 1606, 1509, 1475 (Aromatic

C═C); 1373 (C—N stretching of imide); 1298, 1252 (—O—); 1203, 1167, 1148 (C—F in CF3);

726 (Imide ring deformation).

### Example 1-1

Diamine compounds 0.22114 g (0.658 mmol) prepared (supra) were completely dissolved in 2.6 mL of purified m-cresol and an equivalent amount of 1,2,4,5-benzene tetracarboxylic dianhydride (PMDA) 0.14372 g (0.659 mmol) and a small amount of isoquinoline were added to the solution at room temperature, and the solution was stirred for 4 hours to give the solution containing polyamic acid.

To this solution was added 2.6 mL of m-cresol, and the temperature was raised to 190° C., and a small amount of chlorobenzene was added to remove water produced during imidization, and the solution was stirred for 12 hours.

In this case, the precipitation or gel formation during the reaction was not observed. After cooling to room temperature, the viscous solution was treated with a mixture of methanol and water to promote precipitation, which was washed several times with excess amounts of water and hot methanol, followed by drying in vacuum at 70° C. to obtain polymer.

¹H NMR (DMSO-d₆, 400 MHz, ppm): 8.479 (m, 2H), 8.473 (s, 2H), 7.526 (m, 2H), 7.118 (m, 2H).

Some of the synthesized polymer was made as a 7.5% solution of DMAc by weight which was then coated onto glass plate, followed by placing the glass plate at 100° C. under vacuum for 24 hours to remove the solvent to obtain a transparent and rigid film.

### Example 2

The Example 1 was used to give soluble polyimide, except that 3,3′,4,4′-biphenyl tetracarboxylic dianhydride (BPDA) 0.34793 g (1.183 mmol) was used instead of PMDA, and diamine compound 0.39739 g (1.182 mmol) synthesized from the synthetic example. There was no precipitation or gel formation during reaction.

¹H NMR (DMSO-d₆, 400 MHz, ppm): 8.435 (s, 2H), 8.388 (m, 4H), 8.163 (t, J=7.7 Hz, 1H), 8.086 (t, J=7.6 Hz, 1H), 7.513 (d, J=8.4 Hz, 2H), 7.041 (d, J=8.1 Hz, 2H).

Some of the synthesized polymer was made as a 7.5% solution of DMAc by weight which was then coated onto glass plate, followed by placing the glass plate at 100° C. temperature under vacuum for 24 hours to remove the solvent to obtain a transparent and rigid film.

FTIR (film, cm⁻¹): 1779, 1727 (C═O stretching of imide); 1620, 1509, 1475 (Aromatic C═C); 1383 (C—N stretching of imide); 1298, 1253 (—O—); 1202, 1168, 1146, 1120 (C—F in CF₃); 738 (Imide ring deformation).

### Example 3

The Example 1 was used to give soluble polyimide, except that 3,3′,4,4′-benzophenone tetracarboxylic dianhydride(BTDA) 0.3808 g (1.182 mmol) was used instead of PMDA, and diamine compound 0.3964 g (1.179 mmol) synthesized from the synthetic example. There was no precipitation or gel formation during reaction.

¹H NMR (DMSO-d₆, 400 MHz, ppm): 8.433 (s, 2H), 8.244 (m, 6H), 7.477 (d, J=7.8 Hz, 2H), 7.074 (d, J=8.1 Hz, 2H).

Some of the synthesized polymer was made as a 7.5% solution of DMAc by weight which was then coated onto glass plate, followed by placing the glass plate at 100° C. under vacuum for 24 hours to remove the solvent to obtain a transparent and rigid film.

FTIR (film, cm⁻¹): 1782, 1731 (C═O stretching of imide); 1678 (diaryl ketone of BTDA); 1619-1475 (Aromatic C═C); 1385 (C—N stretching of imide); 1298, 1248 (—O—); 1206, 1164, 1146 (C—F in CF₃); 720 (Imide ring deformation).

### Example 4

The Example 1 was used to give soluble polyimide, except that 3,3′,4,4′-diphenyl ether 2-carboxylic dianhydride(ODPA) 0.36623 g (1.181 mmol) was used instead of PMDA, and diamine compound 0.39648 g (1.179 mmol) synthesized from the synthetic example. There was no precipitation or gel formation during reaction.

¹H NMR (DMSO-d₆, 400 MHz, ppm): 8.395 (s, 2H), 8.146 (t, J=9.4 Hz, 1H), 8.057 (t, J=9.6 Hz, 1H), 7.655 (m, 4H), 7.433 (d, J=7.3 Hz, 2H), 7.041 (d, J=7.7 Hz, 2H).

Some of the synthesized polymer was made as a 7.5% solution of DMAc by weight which was then coated onto glass plate, followed by placing the glass plate at 100° C. under vacuum for 24 hours to remove the solvent to obtain a transparent and rigid film.

FTIR (film, cm⁻¹): 1781, 1727 (C═O stretching of imide); 1608, 1508, 1474 (Aromatic C═C); 1383 (C—N stretching of imide); 1297, 1275, 1253 (—O—); 1201, 1167, 1144, 1113 (C—F in CF₃); 744 (Imide ring deformation).

### Example 5

The Example 1 was used to give soluble polyimide, except that 4,4′-hexafluoro isopropyl polyvinylidene diphthalic anhydride (6-FDA) 0.52558 g) was used instead of PMDA, and diamine compound 0.39624 g (1.178 mmol) prepared from the synthetic example. There was no precipitation or gel formation during reaction.

¹H NMR (DMSO-d₆, 400 MHz, ppm): 8.371 (s, 2H), 8.268 (t, J=9.4 Hz, 1H), 8.170 (t, J=9.4 Hz, 1H), 8.024 (t, J=7.4 Hz, 1H), 7.940 (t, J=7.9 Hz, 1H), 7.804 (d, J=7.0 Hz, 1H), 7.715 (d, J=8.1 Hz, 1H), 7.427 (d, J=8.4 Hz, 2H), 7.051 (d, J=8.4 Hz, 2H).

Some of the synthesized polymer was made as a 7.5% solution of DMAc by weight which was then coated onto glass plate, followed by placing the glass plate at 100° C. under vacuum for 24 hours to remove the solvent to obtain a transparent and rigid film.

FTIR (film, cm⁻¹): 1788, 1735 (C═O stretching of imide); 1509, 1475 (Aromatic C═C); 1385 (C—N stretching of imide); 1298, 1255 (—O—); 1203, 1148, 1121 (C—F in CF₃); 722 (Imide ring deformation).

TABLE 1
Properties of Polyimides

| | | | | | | Temperature of | | Temperature of | | |
|---|---|---|---|---|---|---|---|---|---|---|
| | Tertracarbo | | | | | 5% weight loss | | 10% weight loss | | |
| | xylic acid | Mₙ/10³ (g/ | M_{w}/10³ | | T_{g} | (° C.) | | (° C.) | | CTE |
| | anhydride | mol | (g/mol) | PDI | (° C.) | nitrogen | air | nitrogen | air | (ppm/° C.) |
| Ex. 1 | PMDA | 43.4 | 125.4 | 2.89 | — | 425 | 398 | 543 | 506 | 49.6 |
| Ex. 1-1 | PMDA | 59.9 | 112.5 | 1.88 | — | 570 | 549 | 594 | 575 | 46.1 |
| Ex. 2 | BPDA | 24.6 | 55.1 | 2.24 | 338 | 554 | 536 | 587 | 579 | 54.6 |
| Ex. 3 | BTDA | 27.8 | 64.7 | 2.33 | 315 | 555 | 528 | 588 | 566 | 64.5 |
| Ex. 4 | ODPA | 19.5 | 44.3 | 2.27 | 297 | 508 | 475 | 564 | 551 | 64.3 |
| Ex. 5 | 6-FDA | 20.0 | 42.7 | 2.14 | 319 | 509 | 492 | 542 | 537 | 66.8 |

Mₙ: number-average molecular weight
M_{w}: weight-average molecular weight
PDI: polydispersity Index
T_{g}: glass transition temperature
CTE (Coefficient of Thermal Expansion): TMA was used at the temperature range of 50~250° C.

### Example 1-1

### Polyimide which Underwent Imidation in m-Cresol for 12 Hours

Tg of all synthesized polyimides was above 297° C. shown in Table 1, and in the case of Example 1, Tg is not observed below 500° C. Also, the synthesized polyimide had a higher Tg than the KR 10-0600449 polyimide, which had the diamine monomer (Reference Formula 4) with one trifluoromethyl group.

This is because of the increase in the rigidity of polyimide chain via interfering with the degree of freedom about the ether bond. Also, polyimides after full imidization have excellent thermal stability according to the TGA result, and in case of Example 1, the imidization was not fully complete, therefore, the low heat stability. However, after complete imidization, as in Example 1-1, the resulting polyimide had the best thermal stability, compared to other polyimides.

Also, when compared to examples from KR10-0600449, the thermal expansion efficiency was not highly increased even though one more trifluoromethyl group was present.

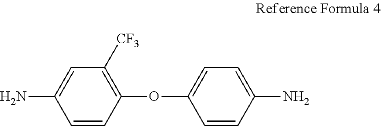 [SMILES: Nc1ccc(Oc2ccc(N)cc2C(F)(F)F)cc1]

TABLE 2
Solubility of Polyimide

| | NMP | DMAc | DMF | DMSO | m-cresol | anisole | THF | chloroform | EA | acetone |
|---|---|---|---|---|---|---|---|---|---|---|
| Ex. 1 | ++ | ++ | ++ | ++ | ++ | ++ | ++ | − | ++ | ++ |
| Ex. 2 | ++ | ++ | ++ | + | ++ | ++ | ++ | +− | − | − |
| Ex. 3 | ++ | ++ | ++ | ++ | ++ | ++ | ++ | −S | −S | −S |
| Ex. 4 | ++ | ++ | ++ | ++ | ++ | ++ | ++ | ++ | +− | +− |
| Ex. 5 | ++ | ++ | ++ | ++ | ++ | ++ | ++ | ++ | ++ | ++ |

* Solubility: ++ soluble at room temperature, + soluble when heated, + − partially soluble,−S swelling, −insoluble.
NMP: N-methyl pyrrolidone
DMAc: N, N-dimethyl acetamide
DMF: N, N-dimethylformamide
DMSO: dimethyl sulfoxide
THF: tetrahydrofuran
EA: ethyl acetate

As shown in Table 2, all polyimides, including Example 1, showed high solubility in polar solvents, such as NMP, DMAc, DMF, DMSO, m-cresol, anisol, and THF, at room temperature. The bulky effect of the trifluoromethyl group as well as the electron withdrawing effect, and low polarization reduce the aggregation between chains, followed by lowering interactions between polyimides.

Examples 4 and 5 are highly soluble in chloroform, and Examples 1 and 5 show good solubility in ethyl acetate and acetone. Among polyimides, Example 5 with the highest concentration of fluorine showed the highest solubility due to the largest interference on the interactions between chains of polyimides. This provides the polyimides of the present invention with good solubility in organic solvent and good processibility after imidation.

As shown in Tables 1 and 2, polyimides synthesized via Scheme 3 using diamine compounds with trifluoromethyl groups substituted asymmetrically had good solubility without reducing heat resistance and thermal expansion coefficients. This is because of the inhibition of several interactions due to the bulky effects of the trifluoromethyl groups, the induction effects and overall asymmetric structure, in spite of the rigid structure due to the introduction of trifluoromethyl groups, thus providing soluble polymer.

TABLE 3
Refractive Index of Polyimide Film

| | | wavelength | | | | | |
|---|---|---|---|---|---|---|---|
| | Thickness (μm) | λ (nm) | nTEa | nTMb | navc | Δnd | εe |
| Ex. 1 | 1.8 | 633 | 1.595 | 1.541 | 1.577 | 0.054 | 2.74 |
| | 2.4 | 1310 | 1.580 | 1.527 | 1.562 | 0.053 | 2.68 |
| Ex. 2 | 6.6 | 633 | 1.627 | 1.563 | 1.606 | 0.064 | 2.84 |
| | 7.1 | 1310 | 1.602 | 1.539 | 1.581 | 0.063 | 2.75 |
| Ex. 3 | 5.0 | 633 | 1.603 | 1.568 | 1.591 | 0.035 | 2.78 |
| | 5.4 | 1310 | 1.586 | 1.547 | 1.573 | 0.039 | 2.72 |
| Ex. 4 | 5.0 | 633 | 1.594 | 1.563 | 1.584 | 0.031 | 2.76 |
| | 6.5 | 1310 | 1.583 | 1.550 | 1.572 | 0.033 | 2.72 |
| Ex. 5 | 4.9 | 633 | 1.545 | 1.517 | 1.536 | 0.028 | 2.60 |
| | 5.4 | 1310 | 1.527 | 1.501 | 1.518 | 0.026 | 2.53 |

* a: the refractive index in the horizontal direction,
b: the refractive index in vertical direction,
c: average refractive index,
d: birefringence,
e: dielectric constant (ε = 1.10 nₐᵥ²) calculated on the basis of average refractive index

As shown in Table 3, the synthesized polyimide has lower birefringence as well as lower refractive index despite the rigid planar structure. This is because of the two bulky trifluoromethyl groups which interfere with the interactions between polyimide chain, and low polarizability due to the fluorine atom in the polyimide, thus enabling the polyimide for applications in electrical and electronic materials.

1: An asymmetric diamine compound having two substituents, R and R′ attached to cyclic group A, represented by Formula 1: 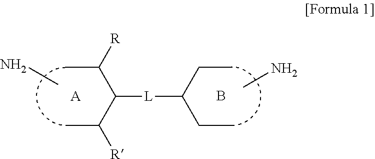
wherein R and R′ each independently represents (i) C1 to C12 alkyl group unsubstituted or substituted with one or more halogen, (ii) C1 to C12 alkoxy group unsubstituted or substituted with one or more halogen, (iii) C2 to C12 alkenyl group unsubstituted or substituted with one or more halogen, (iv) C2 to C12 alkynyl group unsubstituted or substituted with one or more halogen, (v) C4 to C30 cycloalkyl group unsubstituted or substituted with one or more halogen, (vi) cycloalkenyl group unsubstituted or substituted with one or more halogen, (vii) C6 to C30 aryl group unsubstituted or substituted with one or more halogen, C1 to C12 alkyl group, C1 to C12 alkoxy group, C1 to C12 halogenated alkyl group and/or C1 to C12 halogenated alkoxy group, (viii) C3 to C30 heteroaryl group unsubstituted or substituted with one or more halogen, C1 to C12 alkyl group, C1 to C12 alkoxy group, C1 to C12 halogenated alkyl group and/or C1 to C12 halogenated alkoxy group, or (ix) C6 to C30 arylalkyl group unsubstituted or substituted with one or more halogen, C1 to C12 alkyl group, C1 to C12 alkoxy group, C1 to C12 halogenated alkyl group and/or C1 to C12 halogenated alkoxy group;
a linker, ‘-L-’ represents a direct bond, —O—, —S—, —C(═O)O—, —OC(═O)—, —C(═O)—, —SO2-, C(CH3)2-, —C(CF3)2-, —NR″″-, or a combination thereof (where R″″ is hydrogen or C1 to C6 alkyl group unsubstituted or substituted with one or more halogen), and;
said cyclic group 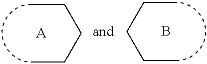
each independently represent (i) C5 to C30 aryl or cycloalkyl group comprising at least one 5-membered or 6-membered ring unsubstituted or substituted with halogen, or (ii) C3 to C30 heteroaryl, or heterocycloalkyl group comprising at least one 5-membered or 6-membered ring unsubstituted or substituted with halogen.

2: The asymmetrical diamine compound according to claim 1, wherein R and R′ are hydrocarbyl group comprising fluorine.

1: The asymmetrical diamine compound according to claim 1, wherein L is any one selected from the group consisting of a direct bond, —O—, and —S—.

4: The asymmetrical diamine compound according to claim 1, represented as Formula 2, wherein two trifluoromethyl groups are attached to one of cyclic group asymmetrically:

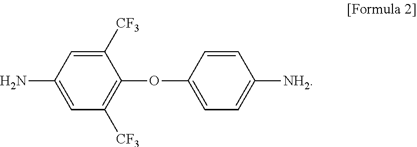 [SMILES: Nc1ccc(Oc2c(C(F)(F)F)cc(N)cc2C(F)(F)F)cc1]

5: A process for producing an asymmetrical diamine compound represented as Formula 1 in Scheme 2, comprising synthesizing dinitro compound represented as Formula C by undergoing nucleophilic substitution reaction between compounds represented as Formula A and Formula B from Scheme 1, followed by hydrogenation of said dinitro compound represented as Formula C: 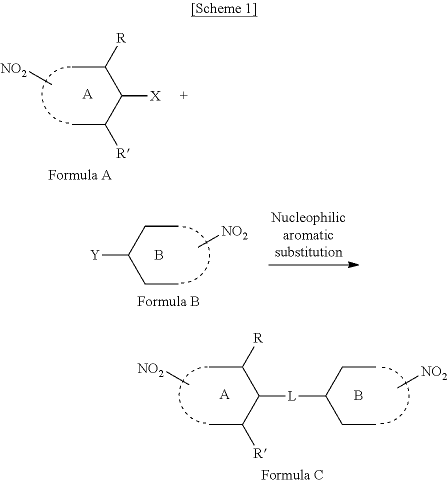 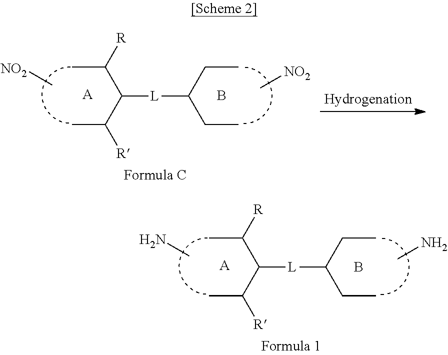
i) wherein, in Scheme 1, one of —X or —Y represents a halogen atom (—F, —Cl, —Br or —I), ester group, —C(O)C1, or sulfonyl chloride, while the other is OH, SH or alkali metal salt thereof which forms -L- linker in dinitro compound of Scheme 1;
ii) wherein R and R′ each independently represents (i) C1 to C12 alkyl group unsubstituted or substituted with one or more halogen, (ii) C1 to C12 alkoxy group unsubstituted or substituted with one or more halogen, (iii) C2 to C12 alkenyl group unsubstituted or substituted with one or more halogen, (iv) C2 to C12 alkynyl group unsubstituted or substituted with one or more halogen, (v) C4 to C30 cycloalkyl group unsubstituted or substituted with one or more halogen, (vi) cycloalkenyl group unsubstituted or substituted with one or more halogen, (vii) C6 to C30 aryl group unsubstituted or substituted with one or more halogen, C1 to C12 alkyl group, C1 to C12 alkoxy group, C1 to C12 halogenated alkyl group and/or C1 to C12 halogenated alkoxy group, (viii) C3 to C30 heteroaryl group unsubstituted or substituted with one or more halogen, C1 to C12 alkyl group, C1 to C12 alkoxy group, C1 to C12 halogenated alkyl group and/or C1 to C12 halogenated alkoxy group, or (ix) C6 to C30 arylalkyl group unsubstituted or substituted with one or more halogen, C1 to C12 alkyl group, C1 to C12 alkoxy group, C1 to C12 halogenated alkyl group and/or C1 to C12 halogenated alkoxy group;
iii) a linker, ‘-L-’ represents a direct bond, —O—, —S—, —C(═O)O—, —OC(═O)—, or —SO₂— and
said cyclic group 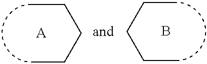
each independently represent (i) C5 to C30 aryl or cycloalkyl group comprising at least one 5-membered or 6-membered ring unsubstituted or substituted with halogen, or (ii) C3 to C30 heteroaryl, or heterocycloalkyl group comprising at least one 5-membered or 6-membered ring unsubstituted or substituted with halogen.

6: The process for producing the asymmetrical diamine compound according to claim 5, wherein R and R′ are hydrocarbyl group comprising fluorine.

7: The process for producing the asymmetrical diamine compound according to claim 5 wherein L is any one selected from the group consisting of a direct bond, —O—, and —S—.

8: The process for producing the diamine compound according to claim 5, wherein the reactants in said Scheme 1,1-bromo-4-nitro-2,6-bis-trifluoromethylbenzene and 4-nitro phenol are used to give dinitro compound, represented as formula 3, followed by hydrogenation of said dinitro compound:

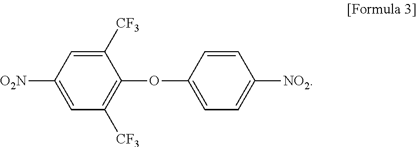 [SMILES: O=[N+]([O-])c1ccc(Oc2c(C(F)(F)F)cc([N+](=O)[O-])cc2C(F)(F)F)cc1]

9: A process for producing polymer selected from a group of polyamide, polyamic acid, and polyimide, wherein asymmetric diamine compound according to claim 1 is used in the polymerization as monomer.

10: The process for producing the polyimide according to claim 9, wherein polyimide, represented as formula 5, is prepared via imidation reaction using said asymmetrical diamine compound and tetracarboxylic acid as monomer; 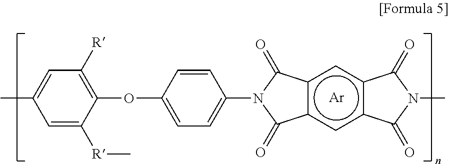
wherein n is an integer selected from 10 to 5,000,000;
Ar is optionally substituted aromatic group, with substituents selected from a group consisting of C1 to C20 alkyl group, or C6 to C20 aryl group, or optionally substituted heterocyclic aromatic group, substituents selected from a group consisting of C1 to C20 alkyl group, or C6 to C20 aryl group; and
R and R′ are the same as defined as claim 1.

11: The A process for producing the polyimide according to claim 10, wherein said asymmetrical diamine compound and tetracarboxylic acid monomers are dissolved in organic solvent to produce polyamic acid, followed by heating and stirring to complete imidization.

12: The process for producing the polyimide according to claim 10, wherein said tetracarboxylic acid is any one selected from a group consisting of 1,2,4,5-Benzenetetracarboxylic dianhydride, 3,3′,4,4′-Benzophenonetetracarboxylic dianhydride, 2,2′,3,3′-Benzophenonetetracarboxylic dianhydride, 3,3′,4,4′-Diphenylethercarboxylic dianhydride, 2,2′,3,3′-Diphenylethercarboxylic dianhydride, 3,3′-Oxydiphthalic dianhydride, 3,3′,4,4′-Biphenyltetracarboxylic dianhydride,
2,2′,3,3′-Biphenyltetracarboxylic dianhydride, diphenyl sulfide-3,3′,4,4′-tetracarboxylic dianhydride, diphenyl sulfide-2,2′,3,3′-tetracarboxylic dianhydride, diphenyl sulfone-3,3′,4,4′-tetracarboxylic dianhydride, diphenyl sulfone-2,2′,3,3′-tetracarboxylic dianhydride, 4,4′-(hexafluoroisopropylidene)diphthalic anhydride, 1,4,5,8-naphthalenetetracarboxylic dianhydride, 1,2,5,6-naphthalenetetracarboxylic dianhydride, 2,3,6,7-naphthalenetetracarboxylic dianhydride.

13: The process for producing the polyimide according to claim 10, wherein the diamine compound is mixed with diamine selected from a group consisting of 4,4′-Diaminodiphenyl ether, 3,4′-Diaminodiphenyl ether, 3,3′-Diaminodiphenyl ether, 2,4′-Diaminodiphenyl ether, 2,2′-Diaminodiphenyl ether, 2,3′-Diaminodiphenyl ether, 1,4-Bis(4-aminophenoxy)benzene, 1,4-Bis(3-aminophenoxy)benzene, 1,3-Bis(4-aminophenoxy)benzene, 1,3-Bis(3-aminophenoxy)benzene, p-Phenylenediamine, m-Phenylenediamine, o-Phenylenediamine, p-Aminobenzylamine, m-Aminobenzylamine, 4,4′-diaminodiphenylmethane, 3,4′-diaminodiphenylmethane, 3,3′-diaminodiphenylmethane, 2,2-Bis[4-(4-aminophenoxyl)phenyl]propane, 2,2-Bis[4-(3-aminophenoxyl)phenyl]propane, Bis(4-aminophenyl)sulfide, Bis(3-aminophenyl)sulfide, 3,4-diaminophenyl sulfide, Bis(4-aminophenyl)sulfoxide, Bis(3-aminophenyl)sulfoxide, 3,4-diaminophenyl sulfoxide, Bis(4-aminophenyl)sulfone, Bis(3-aminophenyl)sulfone, 3,4-diaminophenyl sulfone, 4,4′-diaminobenzophenone, 3,4′-diaminobenzophenone, 3,3′-diaminobenzophenone, Bis[4-(4-aminophenoxyl)phenyl)]sulfone, Bis[4-(3-aminophenoxyl)phenyl)]sulfone,
Bis[4-(4-aminophenoxyl)phenyl)]ether, 1,4-Bis[4-(3-aminophenoxyl)benzoyl]benzene, 1,3-Bis[4-(3-aminophenoxyl)benzoyl]benzene,
4,4′-Bis[3-(4-aminophenoxyl)benzoyl]diphenyl ether, 4,4′-Bis[3-(3-aminophenoxyl)benzoyl]diphenyl ether, 4,4′-Bis[4-(4-amino-α,α-dimethylbenzyl)phenoxy]benzophenone, 4,4′-Bis[4-(4-amino-α,α-dimethylbenzyl)phenoxy]diphenyl sulfone, Bis[4-{4-(4-aminophenoxy)phenoxy}phenyl]sulfone, 1,4-Bis[4-(4-aminophenoxy)-α,α-dimethylbenzyl]benzene, 1,3-Bis[4-(4-aminophenoxy)-α,α-dimethylbenzyl]benzene, 2,2-Bis[4-(4-aminophenoxyl)phenyl]-1,1,1,3,3,3-hexafluoropropane, 2,2-Bis[4-(3-aminophenoxyl)phenyl]-1,1,1,3,3,3-hexafluoropropane, to prepare a polyamic acid.

14: A polymer selected from the group consisting of polyamide, polyamic acid, and polyimide, which is resulting from polymerisation of the asymmetrical diamine according to claim 1.

15: The polyimide according to claim 14, represented as formula 5, which is prepared via imidation reaction using the asymmetric diamine compound according to claim 1 and tetracarboxylic acid as monomer; 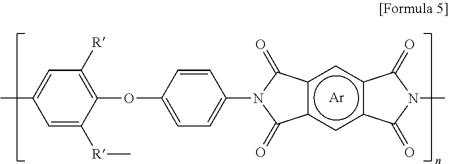
wherein n is an integer selected from 10 to 5,000,000;
Ar is optionally substituted aromatic group, with substituents selected from a group consisting of C1 to C20 alkyl group, or C6 to C20 aryl group, or optionally substituted heterocyclic aromatic group, substituents selected from a group consisting of C1 to C20 alkyl group, or C6 to C20 aryl group; and
R and R′ are the same as defined as claim 1.

16: A polyimide according to claim 15, wherein said tetracarboxylic acid is any one selected from the group consisting of 1,2,4,5-Benzenetetracarboxylic dianhydride, 3,3′,4,4′-Benzophenonetetracarboxylic dianhydride, 2,2′,3,3′-Benzophenonetetracarboxylic dianhydride, 3,3′,4,4′-Diphenylethercarboxylic dianhydride, 2,2′,3,3′-Diphenylethercarboxylic dianhydride, 3,3′-Oxydiphthalic dianhydride, 3,3′,4,4′-Biphenyltetracarboxylic dianhydride, 2,2′,3,3′-Biphenyltetracarboxylic dianhydride, Diphenyl sulfide-3,3′,4,4′-tetracarboxylic dianhydride, Diphenyl sulfide-2,2′,3,3′-tetracarboxylic dianhydride, Diphenyl sulfone-3,3′,4,4′-tetracarboxylic dianhydride, Diphenyl sulfone-2,2′,3,3′-tetracarboxylic dianhydride, 4,4′-(hexafluoroisopropylidene)diphthalic anhydride, 1,4,5,8-naphthalenetetracarboxylic dianhydride, 1,2,5,6-naphthalenetetracarboxylic dianhydride, 2,3,6,7-naphthalenetetracarboxylic dianhydride

17: A polyimide according to claim 15, wherein the diamine compound is mixed with diamine selected from a group consisting of 4,4′-Diaminodiphenyl ether, 3,4′-Diaminodiphenyl ether, 3,3′-Diaminodiphenyl ether, 2,4′-Diaminodiphenyl ether, 2,2′-Diaminodiphenyl ether, 2,3′-Diaminodiphenyl ether, 1,4-Bis(4-aminophenoxy)benzene, 1,4-Bis(3-aminophenoxy)benzene, 1,3-Bis(4-aminophenoxy)benzene, 1,3-Bis(3-aminophenoxy)benzene, p-Phenylenediamine, m-Phenylenediamine, o-Phenylenediamine, p-Aminobenzylamine, m-Aminobenzylamine, 4,4′-diaminodiphenylmethane, 3,4′-diaminodiphenylmethane, 3,3′-diaminodiphenylmethane, 2,2-Bis[4-(4-aminophenoxyl)phenyl]propane, 2,2-Bis[4-(3-aminophenoxyl)phenyl]propane, Bis(4-aminophenyl)sulfide, Bis(3-aminophenyl)sulfide, 3,4-diaminophenyl sulfide, Bis(4-aminophenyl)sulfoxide, Bis(3-aminophenyl)sulfoxide, 3,4-diaminophenyl sulfoxide, Bis(4-aminophenyl)sulfone, Bis(3-aminophenyl)sulfone, 3,4-diaminophenyl sulfone, 4,4′-diaminobenzophenone, 3,4′-diaminobenzophenone, 3,3′-diaminobenzophenone, Bis[4-(4-aminophenoxyl)phenyl)]sulfone, Bis[4-(3-aminophenoxyl)phenyl)]sulfone, Bis[4-(4-aminophenoxyl)phenyl)]ether, 1,4-Bis[4-(3-aminophenoxyl)benzoyl]benzene, 1,3-Bis[4-(3-aminophenoxyl)benzoyl]benzene, 4,4′-Bis[3-(4-aminophenoxyl)benzoyl]diphenyl ether, 4,4′-Bis[3-(3-aminophenoxyl)benzoyl]diphenyl ether, 4,4′-Bis[4-(4-amino-α,α-dimethylbenzyl)phenoxy]benzophenone, 4,4′-Bis[4-(4-amino-α,α-dimethylbenzyl)phenoxy]diphenyl sulfone, Bis[4-{4-(4-aminophenoxyl)phenoxy}phenyl]sulfone, 1,4-Bis[4-(4-aminophenoxy)-α,α-dimethylbenzyl]benzene, 1,3-Bis[4-(4-aminophenoxy)-α,α-dimethylbenzyl]benzene, 2,2-Bis[4-(4-aminophenoxyl)phenyl]-1,1,1,3,3,3-hexafluoropropane, 2,2-Bis[4-(3-aminophenoxyl)phenyl]-1,1,1,3,3,3-hexafluoropropane, to prepare a polyamic acid.

18: A film which is prepared by dissolving the polyimide according to claim 15 in polar aprotic organic solvent or aromatic alcohol.

19: A film according to claim 18, wherein said polar aprotic organic solvent is any one selected from the group consisting of N, N-dimethylformamide (DMF), N, N-dimethyl acetamide (DMAc), N-methyl pyrrolidone (NMP), and dimethyl sulfoxide (DMSO), tetrahydrofuran (THF), and anisole, and said aromatic alcohol includes m-cresol.
